(19) United States
(12) Patent Application Publication
(10) Pub. No.: US 2026/0174523 A1
(43) Pub. Date: Jun. 25, 2026

(54) Bracket Mechanism and Medical Device

(71) Applicant: Dongguan Laiguang Electronic Technology Co., Ltd., Dongguan (CN)

(72) Inventors: Liang WEN, Dongguan (CN); Yinan QIN, Dongguan (CN)

(21) Appl. No.: 19/044,035

(22) Filed: Feb. 3, 2025

(30) Foreign Application Priority Data
Dec. 24, 2024 (CN) 2024232048450

(51) Int. Cl. A61B 90/57; A61B 50/22

(52) U.S. Cl. CPC A61B 90/57; A61B 50/22

(57) ABSTRACT

A bracket assembly includes a first clamping bracket and a second clamping bracket, the first clamping bracket and/or the second clamping bracket are movably provided on a base, a clamping space is formed between the first clamping bracket and the second clamping bracket, and the clamping space is adjusted with the movement of the first clamping bracket or the second clamping bracket. The clamping space is used for clamping a phototherapy component or a physiotherapy component, and the clamping space is compatible with different sizes of the phototherapy component or the physiotherapy component, avoiding clamping only one size of the phototherapy component or the physiotherapy component, thereby improving the versatility of the bracket mechanism.

### CROSS-REFERENCE TO RELATED APPLICATIONS

The present application claims priority to Chinese Patent Application No. 202423204845.0, filed Dec. 24, 2024, the entire disclosure of which is incorporated herein by reference.

### TECHNICAL FIELD

The present application relates to the technical field of medical devices, and particularly relates to a bracket mechanism and a medical device.

### BACKGROUND

With the development of science and technology, the medical equipment is the most basic element of medical treatment, scientific research, teaching, institutions, and clinical disciplines work, i.e., including professional medical equipment, also including household medical equipment, and the bracket mechanism as a part of the medical equipment.

In the related art, the existing bracket mechanism includes a base, a first clamping bracket and a second clamping bracket, the first clamping bracket and the second clamping bracket are arranged relative to each other, the first clamping bracket and the second clamping bracket are connected to the base, and the first clamping bracket and the second clamping bracket are used to clamp a phototherapy component or a physiotherapy component. However, the first clamping bracket and the second clamping bracket cannot be positionally adjusted, and can only clamp a size of the phototherapy component or the physiotherapy component, resulting in poor versatility of the existing bracket mechanism.

### SUMMARY

An object of the present application is to provide a bracket mechanism and a medical device, the bracket assembly includes a first clamping bracket and a second clamping bracket, the first clamping bracket and/or the second clamping bracket are movably provided on a base, and the clamping space is adjusted with the movement of the first clamping bracket or the second clamping bracket. The clamping space is used for clamping a phototherapy component or a physiotherapy component, and the clamping space is compatible with different sizes of the phototherapy component or the physiotherapy component, avoiding clamping only one size of the phototherapy component or the physiotherapy component, thereby improving the versatility of the bracket mechanism. The locking assembly is connected to the first clamping bracket or the second clamping bracket and is used to lock the first clamping bracket or the second clamping bracket to maintain the clamping of the phototherapy component or the physiotherapy component by the first clamping bracket and the second clamping bracket, ensuring the clamping effect of the first clamping bracket and the second clamping bracket on the phototherapy component or the physiotherapy component.

In order to realize the above purpose, the present application provides the following technical solution: a bracket mechanism, including
a base;
a bracket assembly, including a first clamping bracket and a second clamping bracket;
the first clamping bracket and/or the second clamping bracket are removably provided on the base, a clamping space is formed between the first clamping bracket and the second clamping bracket, the clamping space is adjusted with a movement of the first clamping bracket or the second clamping bracket, and the clamping space is configured to clamp a phototherapy component or a physiotherapy component; and
a locking assembly, connected to the first clamping bracket or the second clamping bracket and configured for locking the first clamping bracket or the second clamping bracket to maintain the clamping of the phototherapy component or the physiotherapy component by the first clamping bracket and the second clamping bracket.

In order to realize the above purpose, the present application provides the following technical solution: a medical device including the support mechanism and a medical component, the medical component is connected to the support mechanism, and the medical component is a phototherapy component or a physiotherapy component.

In order to realize the above purpose, the present application provides the following technical solution: a bracket mechanism, including:
a base;
a bracket assembly, including a first clamping bracket and a second clamping bracket,
the first clamping bracket and/or the second clamping bracket are removably provided on the base, a clamping space is formed between the first clamping bracket and the second clamping bracket, the clamping space is adjusted with a movement of the first clamping bracket or the second clamping bracket, and the clamping space is configured to clamp a phototherapy component or a physiotherapy component;
the clamping space is adapted to accommodate different sizes of phototherapy or physiotherapy components; and
the bracket mechanism further includes a plurality of mobile wheels, the plurality of mobile wheels are provided on a lower side of a same base, and the plurality of mobile wheels roll relative to the ground.

Compared with the related art, the beneficial effects of the present application are the bracket assembly includes a first clamping bracket and a second clamping bracket, the first clamping bracket and/or the second clamping bracket are movably provided on a base, a clamping space is formed between the first clamping bracket and the second clamping bracket, and the clamping space is adjusted with the movement of the first clamping bracket or the second clamping bracket. The clamping space is used for clamping a phototherapy component or a physiotherapy component, and the clamping space is compatible with different sizes of the phototherapy component or the physiotherapy component, avoiding clamping only one size of the phototherapy component or the physiotherapy component, thereby improving the versatility of the bracket mechanism. The locking assembly is connected to the first clamping bracket or the second clamping bracket and is used to lock the first clamping bracket or the second clamping bracket to maintain the clamping of the phototherapy component or the physiotherapy component by the first clamping bracket and the second clamping bracket, ensuring the clamping effect of the first clamping bracket and the second clamping bracket on the phototherapy component or the physiotherapy component.

### BRIEF DESCRIPTION OF THE DRAWINGS

In order to more clearly illustrate the technical solutions in the embodiments of the present application, the accompanying drawings to be used in the description of the embodiments will be briefly described below. Obviously, the accompanying drawings in the following description are only some of the embodiments of the present application, and other accompanying drawings may be obtained from these drawings without creative labor for those skilled in the art.

For a more complete understanding of the present application and its beneficial effects, the following description will be given in conjunction with the accompanying drawings. The same accompanying drawing symbols in the following description indicate the same parts.

FIG. 1 is a schematic diagram of a locking state of the bracket mechanism of the present application.

FIG. 2 is another schematic diagram of the locking state of the bracket mechanism of the present application.

FIG. 3 is an exploded view of the locking state of the bracket mechanism of the present application.

FIG. 4 is a schematic diagram of the connection between the first clamping bracket and the locking assembly of the bracket mechanism of the present application.

FIG. 5 is a schematic diagram of an unlocked state of the bracket mechanism of the present application.

### DETAILED DESCRIPTION OF THE EMBODIMENTS

The technical solutions in the embodiments of the present application will be clearly and completely described below in conjunction with the accompanying drawings in the embodiments of the present application. Obviously, the described embodiments are only a part of the embodiments of the present application, and not all of the embodiments. Based on the embodiments in this application, all other embodiments obtained by those skilled in the art without making creative labor fall within the scope of the present application.

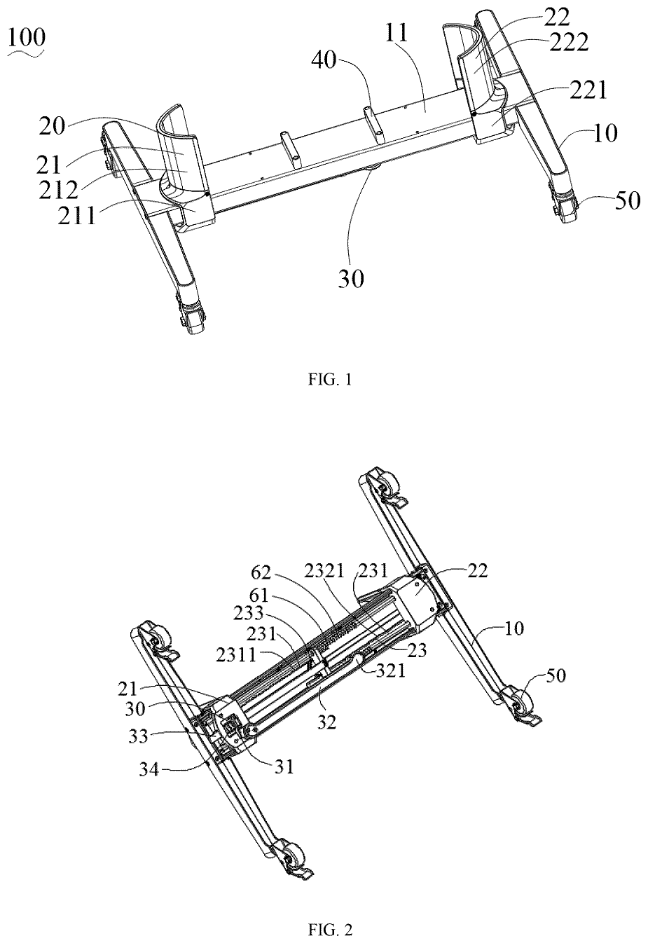

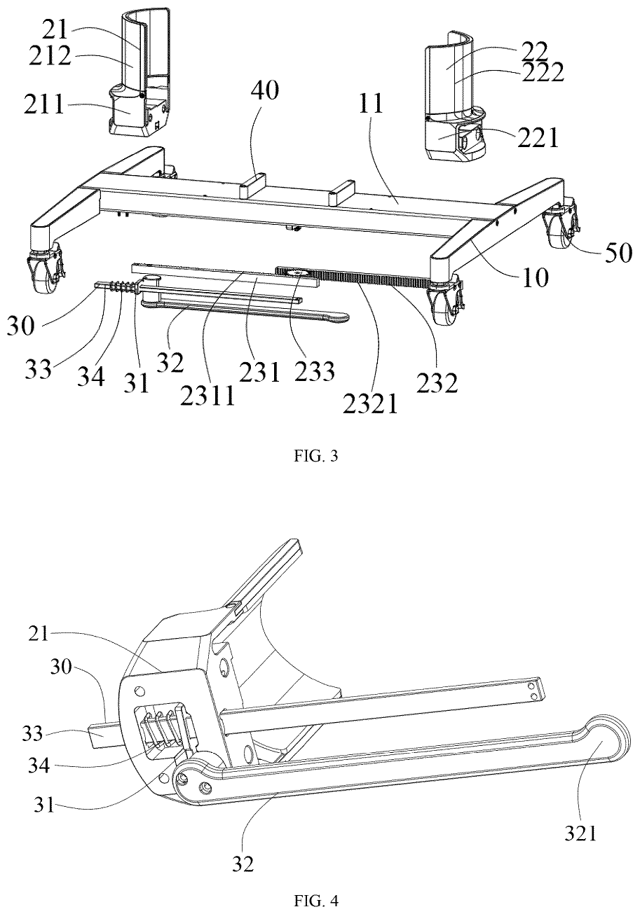

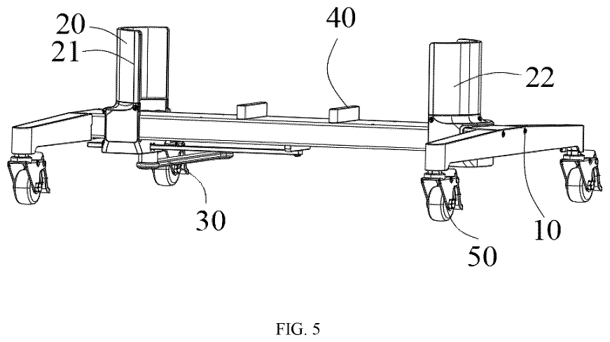

Referring to FIGS. 1˜5, the present application provides a bracket mechanism 100, the bracket mechanism 100 is applied to a medical device, and the bracket mechanism 100 is used to clamp a phototherapy component or a physiotherapy component.

Referring to FIGS. 1˜5, in the present application, the bracket mechanism 100 includes a base 10, a bracket assembly 20 and a locking assembly 30, and the bracket assembly 20 includes a first clamping bracket 21 and a second clamping bracket 22. The first clamping bracket 21 and/or the second clamping bracket 22 are removably provided on the base 10. A clamping space 20a is formed between the first clamping bracket 21 and the second clamping bracket 22, and the clamping space 20a is adjusted as the first clamping bracket 21 or the second clamping bracket 22 move. The clamping space 20a is used for clamping a phototherapy component or a physiotherapy component, and is compatible with different sizes of the phototherapy component or the physiotherapy component, avoiding clamping only one size of the phototherapy component or the physiotherapy component, thereby improving the versatility of the bracket mechanism 100. The locking assembly 30 is connected to the first clamping bracket 21 or the second clamping bracket 22 and is used to lock the first clamping bracket 21 or the second clamping bracket 22 to maintain the clamping of the phototherapy component or the physiotherapy component by the first clamping bracket 21 and the second clamping bracket 22, ensuring the clamping effect of the first clamping bracket 21 and the second clamping bracket 22 on the phototherapy component or the physiotherapy component.

Referring to FIG. 1, in the present application, the base 10 serves as a support component of the bracket mechanism 100, and the base 10 is used to support the bracket assembly 20 and the locking assembly 30.

Referring to FIGS. 1˜3, in the present application, the bracket assembly 20 comprises a first clamping bracket 21 and a second clamping bracket 22, the first clamping bracket 21 and/or the second clamping bracket 22 are movably provided on the base 10 so as to facilitate adjustment of the position of the first clamping bracket 21 and the second clamping bracket 22 relative to the base 10, and a clamping space 20a is formed the first clamping bracket 21 and the second clamping bracket 22, and the clamping space 20a is adjusted with the movement of the first clamping bracket 21 or the second clamping bracket 22. The clamping space 20a is used for clamping a phototherapy component or a physiotherapy component, which is compatible with different sizes of the phototherapy component or the physiotherapy component, avoiding clamping only one size of the phototherapy component or the physiotherapy component, thereby improving the versatility of the bracket mechanism 100.

Referring to FIGS. 1˜4, in the present application, the locking assembly 30 is connected to the first clamping bracket 21 or the second clamping bracket 22 and is used for locking the first clamping bracket 21 or the second clamping bracket 22 to maintain the clamping of the phototherapy component or the physiotherapy component by the first clamping bracket 21 and the second clamping bracket 22, ensuring the clamping effect of the first clamping bracket 21 and the second clamping bracket 22 on the phototherapy component or the physiotherapy component.

Referring to FIGS. 1˜4, when both the first clamping bracket 21 and the second clamping bracket 22 are movably provided on the base 10, a linkage assembly 23 is provided between the first clamping bracket 21 and the second clamping bracket 22, the first clamping bracket 21 and the second clamping bracket 22 are moved closer to each other or farther away from each other driven by the linkage assembly 23, and the clamping space 20a becomes smaller or larger as the first clamping bracket 21 and the second clamping bracket 22 approach or move away from each other, so as to facilitate the clamping space 20a to be compatible with different sizes of phototherapy components or physiotherapy components, avoiding only clamping one size of phototherapy components or physiotherapy components, thereby improving the versatility of the bracket mechanism 100.

Referring to FIGS. 1˜4, the first clamping bracket 21 and the second clamping bracket 22 are arranged relative to each other; the linkage assembly 23 includes a first linkage assembly 231 and a second linkage assembly 232; the first linkage assembly 231 is connected to the first clamping bracket 21; the second linkage assembly 232 is connected to the second clamping bracket 22; the first linkage assembly 231 and the second linkage assembly 232 are connected with each other, such that the linkage of the first clamping bracket 21 relative to the second clamping bracket 22 is realized by the first linkage member 231 and the second linkage member 232, to realize a linkage structure of the first clamping bracket 21 and the second clamping bracket 22.

Referring to FIGS. 1˜3, the first linkage member 231 and the second linkage member 232 are arranged relative to each other; the linkage assembly 23 further includes a connection member 233, the connection member 233 is provided between the first linkage member 231 and the second linkage member 232 and connects the first linkage member 231 and the second linkage member 232, so as to facilitate the connection member 233 to drive the first linkage member 231 and the second linkage member 232 to move simultaneously, such that it is convenient for the connection member 233 to control the first linkage assembly 231 and the second linkage assembly 232, so as to realize that the connection member 233 drives the first clamping bracket 21 and the second clamping bracket 22 via the first linkage assembly 231 and the second linkage assembly 232.

Referring to FIGS. 1˜3, the first linkage assembly 231 is provided with a first rack portion 2311 and the second linkage assembly 232 is provided with a second rack portion 2321. The connection member 233 is a gear, the gear is rotatably connected to the base 10, so as to facilitate adjustment of the position of the gear relative to the base 10, and the gear meshes with the first rack portion 2311 and the second rack portion 2321. The first rack portion 2311 and the second rack portion 2321 move with the rotation of the gears, and the first rack portion 2311 moves inversely with the second rack portion 2321, thereby facilitating the first clamping bracket 21 and the second clamping bracket 22 to move inversely as the first rack portion 2311 and the second rack portion 2321 move inversely.

Referring to FIGS. 1˜3, the base 10 is provided with a crossbar 11; the crossbar 11 is arranged in a horizontal direction, and the first clamping bracket 21 and the second clamping bracket 22 are both provided on the crossbar 11 and move relative to the crossbar 11, so as to facilitate adjustment of the position of the first clamping bracket 21 and the second clamping bracket 22 relative to the crossbar 11, thereby facilitating movement of the first clamping bracket 21 and the second clamping bracket 22 relative to the base 10 via the crossbar 11.

Referring to FIGS. 1˜3, the first clamping bracket 21 includes a first mobile seat 211 and a first clamping portion 212, the first clamping portion 212 is provided on an upper side of the first mobile seat 211, the first clamping portion 212 is connected to the first mobile seat 211 and is provided on an outside of the crossbar 11, so as to facilitate full utilization of the outer space of the crossbar 11 by the first clamping portion 212, and the first clamping portion 212 is used to clamp a side of the phototherapy component or the physiotherapy component. The first mobile seat 211 is socketed to the crossbar 11 and moves relative to the crossbar 11, so as to facilitate adjustment of the position of the first mobile seat 211 relative to the crossbar 11, the first clamping portion 212 moves with the movement of the first mobile seat 211, and the first clamping bracket 21 moves relative to the crossbar 11 by the first mobile seat 211.

Referring to FIGS. 1˜3, the second clamping bracket 22 includes a second mobile seat 221 and a second clamping portion 222, the second clamping portion 222 is provided on an upper side of the second mobile seat 221, the second clamping portion 222 is connected to the second mobile seat 221 and is provided on the outside of the crossbar 11, so as to facilitate the full utilization of the outer space of the crossbar 11 by the second clamping portion 222, the second clamping portion 222 is used to clamp another side of the phototherapy component or the physiotherapy component. The second mobile seat 221 is socketed to the crossbar 11 and moves relative to the crossbar 11, so as to facilitate adjustment of the position of the second mobile seat 221 relative to the crossbar 11, the second clamping portion 222 moves along with the movement of the second mobile seat 221, and the second clamping bracket 22 moves relative to the crossbar 11 by the second mobile seat 221.

Referring to FIGS. 1˜3, the first clamping portion 212 and the second clamping portion 222 are shaped of circular arc and adapted to the phototherapy component or the physiotherapy component, so as to facilitate the adaptation of the inner wall of the first clamping portion 212 and the inner wall of the second clamping portion 222 to the outer wall of the phototherapy component or the physiotherapy component, which ensures the clamping effect of the first clamping portion 212 and the second clamping portion 222 relative to the phototherapy component or the physiotherapy component.

Referring to FIGS. 1˜3, the support mechanism 100 further includes a support seat 40, the support seat 40 is provided on the upper side of the base 10, the support seat 40 is connected to the base 10 and is provided between the first clamping bracket 21 and the second clamping bracket 22. The support seat 40 is used to support the bottom of the phototherapy component or the bottom of the physiotherapy component, such that there is a gap between the bottom of the phototherapy component or the bottom of the physiotherapy component and the base 10, so as to facilitate the first clamping bracket 21, the second clamping bracket 22 and the support seat 40 to limit the phototherapy component or the physiotherapy component to different positions, ensuring the stability of the phototherapy component or the physiotherapy component relative to the base 10, and avoiding the phototherapy component or the physiotherapy component from shaking.

Referring to FIGS. 1˜3, the support mechanism 100 further includes a plurality of mobile wheels 50, the plurality of mobile wheels 50 are provided on a lower side of the base 10 and connected to the same base 10, the plurality of mobile wheels 50 roll relative to the ground, so as to facilitate the movement of the support mechanism 100 relative to the ground through the plurality of mobile wheels 50, to improve the ease of transferring the support mechanism 100, and increase the stability of movement of the base 10 relative to the ground by the plurality of mobile wheels 50. Optionally, the mobile wheels 50 are brake-type mobile wheels 50 and can be stationary under the action of an external force so as to enable the bracket mechanism 100 to be stationary relative to the ground, avoiding an accidental deflection of the bracket mechanism 100 relative to the ground.

Referring to FIGS. 1˜4, the locking assembly 30 includes a locking member 31; when the locking assembly 30 is connected to the first clamping bracket 21, the locking member 31 is connected to the first clamping bracket 21 and locks the first clamping bracket 21 and the base 10, such that the first clamping bracket 21 is in a locking state relative to the base 10, so as to allow the first clamping bracket 21 to be in a stationary state relative to the base 10, avoiding the accidental adjustment of the clamping space 20a, which ensures the clamping effect of the first clamping bracket 21 relative to the phototherapy component or the physiotherapy component.

Referring to FIGS. 1˜4, the locking assembly 30 further includes a handle 32 and a connection rod 33, the connection rod 33 is connected to the base 10; the handle 32 is provided between the first clamping bracket 21 and the locking member 31; the handle 32 is swingably connected to the first clamping bracket 21, so as to facilitate the adjustment of the handle 32 relative to the first clamping bracket 21, and the handle 32 drives the locking member 31 to be snapped on the connection rod 33, such that the first clamping bracket 21 is in a locking state relative to the base 10, so as to facilitate control of the locking state of the locking member 31 by the handle 32, the locking convenience of the first clamping bracket 21 relative to the base 10 is improved.

Referring to FIGS. 1˜4, the locking member 31 is provided on the outside of the connection rod 33, and the locking member 31 is adjusted with the swinging of the handle 32 relative to the first clamping bracket 21 and is snapped to or loosened from the connection rod 33, and the locking member 31 is snapped to the connection rod 33 to limit the position of the connection rod 33, so as to facilitate the connection rod 33 limiting the first clamping bracket 21, the first clamping bracket 21 is in a locked state relative to the base 10, ensuring that the clamping space 20a remains unchanged.

Referring to FIG. 4, when the locking member 31 is loosened from the connection rod 33, so as to facilitate the connection rod 33 to be movable, the first clamping bracket 21 is in a free state relative to the base 10 and is movable relative to the base 10, such that the first clamping bracket 21 is in an unlocked state relative to the base 10, thereby facilitating the adjustment of the size of the clamping space 20a.

Referring to FIG. 4, the locking assembly 30 further includes a first elastic member 34, the first elastic member 34 is provided between the base 10 and the locking member 31, and exerts an elastic force on the locking member 31, such that the locking member 31 is further snapped to the connection rod 33 under the elastic force of the first elastic member 34, which ensures the locking effect of the locking member 31 relative to the connection rod 33, and prevents the locking member 31 from being accidentally unlocked relative to the connection rod 33.

Referring to FIG. 4, the first elastic member 34 is socketed to the connection rod 33 and is provided on an outside of the connection rod 33, two ends of the first elastic member 34 are in elastic contact with the base 10 and the locking member 31 respectively, and the base 10 and the locking member 31 compress the first elastic member 34, with the base 10 acting as a fixing member and the locking member 31 acting as a movable member, so as to facilitate the first elastic member 34 exerting the elastic force on the locking member 31, to realize a locking effect of the locking member 31 relative to the connection rod 33.

Referring to FIG. 4, one end of the handle 32 away from the first clamping bracket 21 is the grasping end 321, the grasping end 321 is available for the user to grasp, and the user grasps the grasping end 321 to swing the handle 32, which improves the operating comfort of the handle 32.

Optionally, the bracket mechanism 100 further includes a guide rod 61 and a second elastic member 62, the guide rod 61 is connected to the first clamping bracket 21 and passed through and provided on the base 10. The second elastic member 62 is connected between the guide rod 61 and the base 10, and exerts an elastic force to the guide rod 61 to limit a minimum distance between the first clamping bracket 21 and the second clamping bracket 22. Optionally, the second elastic member 62 is a compression spring, and The elastic force exerted on the guide rod 61 by the second elastic member 62 limits the minimum distance between the first clamping bracket 21 and the second clamping bracket 22, it is ensured that the minimum distance between the first clamping bracket 21 and the second clamping bracket 22 is limited, and the minimum distance between the first clamping bracket 21 and the second clamping bracket 22 is greater than 5 mm. When the handle 32 is operated, the first clamping bracket 21 moves 0-5 mm with the swing of the handle 32, ensuring that the handle 32 can still drive further locking of the first clamping bracket 21 within the minimum distance between the first clamping bracket 21 and the second clamping bracket 22, and avoiding damage to the handle by the user exerting excessive force on the handle.

In another embodiment, a medical device includes a bracket mechanism 100 and a medical component, the medical component is connected to the bracket mechanism 100, such that the medical component is connectable to or detachable from the bracket mechanism 100, so as to facilitate the replacement of the medical component with different functions, which improves the versatility of the medical device, and optionally, the medical component is a phototherapy component or the physiotherapy component.

Compared with the related art, the beneficial effect of the present application is:

The present application provides a bracket mechanism 100 and a medical device, and the bracket assembly 20 includes a first clamping bracket 21 and a second clamping bracket 22. The first clamping bracket 21 and/or the second clamping bracket 22 are removably provided on the base 10. A clamping space 20a is formed between the first clamping bracket 21 and the second clamping bracket 22, and the clamping space 20a is adjusted as the first clamping bracket 21 or the second clamping bracket 22 moves. The clamping space 20a is used for clamping a phototherapy component or a physiotherapy component, and is compatible with different sizes of the phototherapy component or the physiotherapy component, avoiding clamping only one size of the phototherapy component or the physiotherapy component, thereby improving the versatility of the bracket mechanism 100. The locking assembly 30 is connected to the first clamping bracket 21 or the second clamping bracket 22 and is used to lock the first clamping bracket 21 or the second clamping bracket 22 to maintain the clamping of the phototherapy component or the physiotherapy component by the first clamping bracket 21 and the second clamping bracket 22, ensuring the clamping effect of the first clamping bracket 21 and the second clamping bracket 22 on the phototherapy component or the physiotherapy component.

In the above embodiments, the description of each embodiment has its own focus, and the parts that are not detailed in a certain embodiment can be seen in the relevant description of other embodiments.

In the description of the present application, the terms “first” and “second” are used for descriptive purposes only, and are not to be understood as indicating or implying relative importance or implicitly specifying the number of technical features indicated. As a result, a feature defined with “first” or “second” may include one or more features, either explicitly or implicitly.

Herein adopts specific examples for elaboration of the principle and implementation of the present application, the illustration of the above examples are only used to help understand the method and the core ideas of the present application; and the those skilled in the art, based on the idea of the present application, will make some change in the specific implementation of the application and the scope of the application, in summary, the contents of this specification should not be construed as a limitation on the present application.

What is claimed is:

1. A bracket mechanism, comprising:
a base;
a bracket assembly, comprising a first clamping bracket and a second clamping bracket,
wherein the first clamping bracket and/or the second clamping bracket are removably provided on the base, a clamping space is formed between the first clamping bracket and the second clamping bracket, the clamping space is adjusted with a movement of the first clamping bracket or the second clamping bracket, and the clamping space is configured to clamp a phototherapy component or a physiotherapy component; and
a locking assembly, connected to the first clamping bracket or the second clamping bracket and configured for locking the first clamping bracket or the second clamping bracket to maintain the clamping of the phototherapy component or the physiotherapy component by the first clamping bracket and the second clamping bracket.

2. The bracket mechanism according to claim 1, wherein when the first clamping bracket and the second clamping bracket are movably provided on the base, a linkage assembly is provided between the first clamping bracket and the second clamping bracket, and the first clamping bracket and the second clamping bracket are moved closer to each other or further away from each other driven by the linkage assembly.

3. The bracket mechanism according to claim 2, wherein the first clamping bracket and the second clamping bracket are arranged opposite each other;
the linkage assembly comprises a first linkage member and a second linkage member;
the first linkage member is connected to the first clamping bracket;
the second linkage member is connected to the second clamping bracket; and
the first linkage member and the second linkage member are connected to each other to realize a linkage structure of the first clamping bracket and the second clamping bracket.

4. The bracket mechanism according to claim 3, wherein the first linkage member and the second linkage member are arranged opposite each other;
the linkage assembly further comprises a connection member, the connection member is provided between the first linkage member and the second linkage member and connect the first linkage member and the second linkage member.

5. The bracket mechanism according to claim 1, wherein the base is provided with a crossbar;
the first clamping bracket and the second clamping bracket are both provided on the crossbar and movable relative to the crossbar;
the first clamping bracket comprises a first mobile seat and a first clamping portion, and the first clamping portion is connected to the first mobile seat and provided on an outside of the crossbar, and the first mobile seat is socketed to the crossbar and is movable relative to the crossbar;
the second clamping bracket comprises a second mobile seat and a second clamping portion, the second clamping portion is connected to the second mobile seat and provided on the outside of the crossbar; and
the second mobile seat is socketed to the crossbar and is movable relative to the crossbar, and the first clamping portion and the second clamping portion are shaped of circular arc and adapted to the phototherapy component or the physiotherapy component.

6. The bracket mechanism according to claim 1, wherein the locking assembly comprises a locking member;
when the locking assembly is connected to the first clamping bracket, the locking member is connected to the first clamping bracket and locks the first clamping bracket to the base, such that the first clamping bracket is in a locked state relative to the base.

7. The bracket mechanism according to claim 6, wherein the locking assembly further comprises a handle and a connection rod, and the connection rod is connected to the base;
the handle is provided between the first clamping bracket and the locking member;
the handle is swingably connected to the first clamping bracket and drives the locking member to snap onto the connection rod, such that the first clamping bracket is in a locked state relative to the base; and
the locking member is provided on an outside of the connection rod, the locking member is adjusted with a swinging of the handle relative to the first clamping bracket, and snapped on or loosened from the connection rod.

8. The bracket mechanism according to claim 7, wherein the locking assembly further comprises a first elastic member, the first elastic member is provided between the base and the locking member and exerts an elastic force on the locking member, such that the locking member is further snapped to the connection rod by the elastic force of the first elastic member.

9. The bracket mechanism according to claim 1, wherein the bracket mechanism further comprises a guide rod and a second elastic member;
the guide rod is connected to the first clamping bracket and passed through and provided on the base;
the second elastic member is connected between the guide rod and the base and exerts an elastic force on the guide rod.

10. The bracket mechanism according to claim 3, wherein the first linkage member is provided with a first rack portion and the second linkage member is provided with a second rack portion; wherein the linkage member is a gear, the gear is rotatably connected to the base, the gear engages the first rack portion and the second rack portion, and the first rack portion and the second rack portion move with a rotation of the gear, and the first rack portion moves in an opposite direction to the second rack portion.

11. The bracket mechanism according to claim 1, wherein the bracket mechanism further comprises a support seat, the support seat is provided on an upper side of the base, the support seat is connected to the base and provided between the first clamping bracket and the second clamping bracket;
wherein the support seat is configured to support a bottom of the phototherapy component or a bottom of the physiotherapy component, such that there is a gap between the bottom of the phototherapy component or the bottom of the physiotherapy component and the base.

12. The bracket mechanism according to claim 1, wherein the bracket mechanism further comprises a plurality of mobile wheels, the plurality of mobile wheels are provided on a lower side of a same base, the plurality of mobile wheels roll relative to the ground.

13. The bracket mechanism according to claim 12, wherein the mobile wheels are brake-type mobile wheels and are capable of being stationary under an external force, so that the bracket mechanism is immobilized relative to the ground.

14. A medical device, comprising the bracket mechanism as claimed in claim 1 and a medical component, wherein the medical component is connected to the bracket mechanism, and the medical component is the phototherapy component or the physiotherapy component.

15. A bracket mechanism, comprising:
a base;
a bracket assembly, comprising a first clamping bracket and a second clamping bracket,
wherein the first clamping bracket and/or the second clamping bracket are removably provided on the base, a clamping space is formed between the first clamping bracket and the second clamping bracket, the clamping space is adjusted with a movement of the first clamping bracket or the second clamping bracket, and the clamping space is configured to clamp a phototherapy component or a physiotherapy component;
wherein the clamping space is adapted to accommodate different sizes of phototherapy or physiotherapy components; and
wherein the bracket mechanism further comprises a plurality of mobile wheels, the plurality of mobile wheels are provided on a lower side of a same base, and the plurality of mobile wheels roll relative to the ground.

16. The bracket mechanism according to claim 15, wherein, wherein the first clamping bracket and the second clamping bracket are both movably provided to the base, a linkage assembly is provided between the first clamping bracket and the second clamping bracket, and the first clamping bracket and the second clamping bracket are driven by the linkage assembly to move closer to each other or further away from each other.

17. The bracket mechanism according to claim 16, wherein the first clamping bracket and the second clamping bracket are arranged opposite each other;
wherein the linkage assembly comprises a first linkage member and a second linkage member;
the first linkage member is connected to the first clamping bracket;
the second linkage is connected to the second clamping bracket; and
the first linkage member and the second linkage member are connected to realize a linkage structure of the first clamping bracket and the second clamping bracket.

18. The bracket mechanism according to claim 15, wherein the base is provided with a crossbar;
wherein the first clamping bracket and the second clamping bracket are both provided on the crossbar and movable relative to the crossbar, the first clamping bracket comprises a first mobile seat and a first clamping portion, the first clamping portion is connected to the first mobile seat and is provided on an outside of the crossbar, and the first mobile seat is socketed to the crossbar and moves relative to the crossbar;
wherein the second clamping bracket comprises a second mobile seat and a second clamping portion, the second clamping portion is connected to the second mobile seat and is provided on the outside of the crossbar; the second mobile seat is socketed to the crossbar and is movable relative to the crossbar; and
the first clamping portion and the second clamping portion both are shaped of circular arc arrangement and adapted to the phototherapy component or the physiotherapy component.

19. The bracket mechanism according to claim 15, wherein the bracket mechanism further comprises a guide rod and a second elastic member;
the guide rod is connected to the first clamping bracket and passed through and provided on the base; and
the second elastic member is connected between the guide rod and the base and exerts an elastic force on the guide rod.

20. The bracket mechanism according to claim 11, wherein the bracket mechanism further comprises a locking assembly, the locking assembly is connected to the first clamping bracket or the second clamping bracket for locking the first clamping bracket or the second clamping bracket to maintain a clamping of the first clamping bracket and the second clamping bracket on the phototherapy component or the physiotherapy component.
